(19) United States
(12) Patent Application Publication
(10) Pub. No.: US 2004/0194352 A1
(43) Pub. Date: Oct. 7, 2004

(54) Orthopedic insole for a diabetic shoe

(76) Inventors: Todd D. Campbell, Petaluma, CA (US); Russell C. Davis, Greenbrae, CA (US); William Y. Guthrie, Fairfax, CA (US)

Correspondence Address: JOHNSON & STAINBROOK, LLP, 3558 ROUND BARN BLVD., SUITE 203, SANTA ROSA, CA, 95403, US

(21) Appl. No.: 10/409,550

(22) Filed: Apr. 7, 2003

(51) Int. Cl. A61F 5/14

(52) U.S. Cl. 36/174; 36/166

(57) ABSTRACT

The invention provides an orthopedic insole for a diabetic shoe, as well as a method of manufacturing an orthopedic insole for a diabetic shoe. The orthopedic insole includes a cupped heel portion, the cupped heel portion having a concave upper bearing surface that extends above a most posterior cephalad portion of a calcaneous; a mid-foot portion continuously coupled to the cupped heel portion, the mid-foot portion having a medial longitudinal arch and a curvilinear upper bearing surface; and a heat-malleable forefoot portion coupled to the mid-foot portion, the forefoot portion having a heat-deformable upper bearing surface.

### CROSS REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present invention relates generally to orthopedic devices, and more particularly to an orthopedic insole for a shoe used to abate and prevent foot and related ailments commonly suffered by diabetics.

### BACKGROUND INFORMATION AND DISCUSSION OF RELATED ART

Diabetes mellitus is a chronic disease that affects the lives of about sixteen million people in the United States, with approximately 780,000 diabetics newly identified each year. It is estimated that 25 percent of all diabetics will develop a serious foot condition at some time in their lives. While around 60,000 foot amputations are performed on people with diabetes each year in the United States, an estimated 50,000 per year could be prevented through the use of diabetic footwear.

Diabetic footwear and other orthopedic devices help reduce the risk of many diabetes-related foot complications, such as callus formation, foot ulceration, poor circulation, loss of feeling, decreased resistance to infection, foot deformity, and neuropathy.

Neuropathy, a nervous system impairment that affects about 60 to 70 percent of people with diabetes, can cause loss of feeling in feet that increases the risk of undetected injury. Diabetics suffering from neuropathy can develop minor cuts, scrapes, blisters, or pressure sores that they may be completely unaware of due to the insensitivity. If these minor injuries are left untreated, complications may result and lead to ulceration, gangrene, and possibly even amputation.

A diabetic person also may develop deformities and difficulties of the foot such as bunions, hammer toe, Charcot feet, collapsed joints, and motor weakness. The severely deformed foot may no longer have a normal bottom or plantar surface of the foot and may develop abnormal prominence on the plantar or weight-bearing surface of the foot that is subject to increased pressure along with vertical and shearing forces. Increased localized pressure and the loss of the ability to feel pain have been implicated in the development of foot ulcers, which can progress to infection, gangrene, and potential loss of limb.

Maximizing functional control of the foot with the use of a foot support while reducing motion between the foot, the support and the shoe, helps improve foot function, reduce the risk of developing deformities, and effectively slow down the progression of a deformity. Also, a support may help to prevent recurrence of deformity post-operatively. Additionally, there are many disease processes that can affect the foot leading to severe foot deformities, loss of or diminished sensation in the feet, as well as affecting other vital structures of the foot.

Protective footwear is considered one of the best ways to prevent the aforementioned problems of diabetics. Currently, there are orthopedic shoe inserts and insoles that work to give a more even weight distribution and take pressure off of sore spots such as the ball of the foot, corns in between toes, and bunions.

Conventional shoe inserts or soles, which may consist of materials such as elastomeric foam with an impact cushioning foam layer or space filler, usually provide insufficient foot protection. The top surface of the contoured foam material does not to provide enough protection or support for proper biomechanical functions of the foot, particularly in the rearfoot and arch areas. Thus, they are often ineffective in preventing or alleviating foot conditions experienced by diabetics or people with other severe foot problems.

Custom orthopedic devices for diabetics often have a relatively rigid, resilient base comprising a heel portion and an arch portion, contoured to fit the plantar or bottom surface of the foot. They are generally created from hard plastics by using a mold after extensive measurements of a foot, and modified as needed to provide prescribed corrections by a podiatrist. Unfortunately, most customized prescription orthopedic shoes for diabetics require generation of molds for the feet and fabrication of the devices with a delay of several weeks between the taking of measurements for an orthopedic shoe and the arrival of the new customized shoes.

With regard to custom-fitting footwear, there have been a number of approaches. In one approach, a chemical reaction is initiated in a formable material in a footbed, the person then steps into the footwear or shoe and forms an impression, and the material is allowed to cure before the footbed is used. U.S. Pat. No. 3,968,577 illustrates a system in which an impression of the foot is made, and the material is cured or set either pursuant to room temperature vulcanizing or by being heated in an oven for a long period of time. Other patents disclose a shoe or sandal having a bottom layer of a thermoplastic material, which is softened by heat. A person steps into the shoe and an impression of a foot is made in the heated thermoplastic material, which retains the impression of the foot after being cooled.

Various foot orthopedic devices using material formable by chemical reaction or heat application are described in U.S. Pat. No. 3,325,919 by Robinson; U.S. Pat. No. 3,641,688 by von den Benken; U.S. Pat. No. 3,895,405 by Edwards; U.S. Pat. No. 3,968,577 by Jackson; U.S. Pat. No. 4,128,951 by Tansill; U.S. Pat. No. 4,413,429 by Power; U.S. Pat. No. 4,428,089 by Dawber et al.; U.S. Pat. No. 4,433,494 by Courvoisier et al.; U.S. Pat. No. 4,463,761 by Pols et al.; U.S. Pat. No. 4,503,576 by Brown; U.S. Pat. No. 4,510,636 by Phillips; U.S. Pat. No. 4,520,581 by Irwin et al.; U.S. Pat. No. 4,674,206; DeBettignies U.S. Pat. No. 4,868,945 by DeBettingnies; U.S. Pat. No. 4,888,225 by Sandvig et al.; U.S. Pat. No. 4,901,390 by Daley; U.S. Pat. No. 5,101,580 by Lyden; and U.S. Pat. No. 5,203,793 by Lyden.

Unfortunately, if these processes do not result in a proper fit the first time, the insole cannot be remolded and must be discarded. Moreover, they generally require expensive equipment and footwear designs as well as time-consuming production efforts. Many of the molding methods involve the injection of moldable and sometimes chemically reactive material around the foot and/or the application of heat to the material surrounding the foot. A description of such materials and methods can be found in U.S. Pat. No. 5,555,584 by Moore, et al; U.S. Pat. No. 5,632,057 by Lyden; U.S. Pat. No. 5,714,098 by Potter; U.S. Pat. No. 5,733,647 by Moore et al; U.S. Pat. No. 5,879,725 by Potter; U.S. Pat. No. 6,025,414 by Rich; U.S. Pat. No. 6,195,917 by Dieckhaus; U.S. Pat. No. 6,280,815 by Ersfeld et al; and U.S. Pat. No. 6,412,194 by Carlson et al.

A further problem with in-situ shoe molding is that there is no allowance for the orthopedic devices to compensate for foot problems such as the tendency to over-pronate or supinate. U.S. Pat. No. 5,829,171 by Weber et al, discloses a prefabricated heat-softenable insole with a built-in electric heater or heat member, yet the insole is limited in its ability to change shape and provides limited orthopedic benefit.

An insole for a protective diabetic shoe needs to conform to deformities of the foot, while additionally controlling the subtalor joint and realigning the foot and anklebones to their neutral position. One such unitary orthotic device that is designed for significant control of foot motion and realignment and helps prevent excessive foot pronation is disclosed in U.S. patent application Ser. No. 2002/0162250, entitled “Unitary Orthotic Insert and Orthopedic Insole” by Guthrie et al., filed Nov. 7, 2002, the contents of which are hereby incorporated by reference.

An improved diabetic insole would be individually fit to the diabetic foot, thereby reducing any concentrated stress on the foot and the potential for pressure related ailments. The device should be a full-length and full-width protective insole sized to accommodate the entire undersurface of a diabetic foot. Additionally, the device would be adaptable to many types and sizes of adult and children's shoes while needing only a limited number of orthotic insert blanks to fit and be useful in a variety of work, sport, dress, and other shoes. An improved orthopedic insert would include a material that is readily softened and may be re-softened when an initial fit is unsuccessful. It could be fit into a shoe during a single office visit to a medical foot specialist.

Therefore, an object of this invention provides a customizable insole and diabetic shoe that overcome the deficiencies and obstacles described above. Another objective of this invention provides a customizable diabetic shoe that may be fit and completed during one office visit. Additionally the custom orthopedic insole and diabetic shoe would have the ability to control the subtalor joint and realign the foot and anklebones to their neutral position, and provide preventative and curative properties for a variety of foot ailments, particularly for individuals with diabetes mellitus.

### BRIEF SUMMARY OF THE INVENTION

One aspect of the invention provides an orthopedic insole for a diabetic shoe. The orthopedic insole includes a cupped heel portion, the cupped heel portion having a concave upper bearing surface that extends above a most posterior cephalad portion of a calcaneous. In addition, the orthopedic insole has a mid-foot portion continuously coupled to the heel portion, the mid-foot portion having a medial longitudinal arch and a curvilinear upper bearing surface, and a heat-malleable forefoot portion coupled to the mid-foot portion, the forefoot portion having a heat-deformable upper bearing surface.

Other aspects of the invention include a diabetic shoe with an orthopedic insole, and a method of manufacturing an orthopedic insole for a diabetic shoe. The method of manufacturing an orthopedic insole for a diabetic shoe includes providing an orthopedic insole mold with a cavity for a cupped heel portion, a mid-foot portion, and a forefoot portion, the forefoot portion having a heat-deformable upper bearing surface. An injection-molding compound is injected into the orthopedic insole mold and the orthopedic insole is released from the orthopedic insole mold.

### BRIEF DESCRIPTION OF THE SEVERAL VIEWS OF THE DRAWINGS

The following drawings are shown with left feet, left orthopedic insoles, and left shoes, and it should be understood that the right foot, insoles and shoes are substantially mirror images of the left side. It should also be understood that the use of the word shoe, in the context of this document, is intended to be synonymous with nearly all articles of footwear, including but not limited to boots, sandals, open-toe shoes and closed-toe shoes. Footwear also includes socks for diving suits, swimming flippers, water and snow ski boots, and skates such as ice skates and inline skates.

Characteristics and advantages of the invention will become apparent from the following detailed descriptions of particular but not exclusive embodiments, illustrated by way of non-limitative examples in the accompanying drawings, wherein:

FIG. 1 illustrates a side view and a top view of a human foot;

FIG. 2 illustrates a perspective view of a foot and diabetic shoe, the latter assembled with a two-thirds or partial-length pre-molded insole portion and a heat-malleable forefoot portion, in accordance with the current invention;

FIG. 3 illustrates a perspective view of a foot and diabetic shoe, the latter assembled with a full-length pre-molded insole portion and a full-length heat-malleable insole portion, in accordance with the current invention;

FIG. 4 illustrates a perspective view of a full-length pre-molded portion of an orthopedic insole for a diabetic shoe, in accordance with the current invention;

FIG. 5 illustrates a cross-sectional view of an orthopedic insole with a heat-malleable forefoot portion and a pre-molded piece of two-thirds length with mid-foot and cupped heel portions, in accordance with the current invention;

FIG. 6 illustrates a perspective view of an inside of a full-length orthopedic insole for a diabetic shoe, in accordance with the current invention; and

FIG. 7 is a flow diagram of a method of manufacturing an orthopedic insole for a diabetic shoe, in accordance with the current invention.

### DETAILED DESCRIPTION OF THE INVENTION

The orthopedic insole for a diabetic shoe of the present invention provides a customizable insole and diabetic shoe that have the ability to control the subtalor joint and realign the foot and anklebones to their neutral position, and provide preventative and curative properties for a variety of foot ailments, particularly for those with diabetes mellitus. The insole protects the underside of a user's foot, limits the stretching of the plantar fascia, and stabilizes the heel of the foot while a person is walking or running. The cupped heel portion, the mid-foot portion, and the forefoot portion of the orthopedic insole cooperate to provide a therapeutic characteristic for a podiatric condition. The orthopedic insole for a diabetic shoe may be fit and completed during one office visit to a podiatric specialist.

The diabetic insole incorporates a heat-malleable material that may be individually fit to the diabetic foot, thereby reducing any concentrated stress on the foot and the potential for pressure related ailments. The device may be a partial or full-length and full-width protective insole sized to accommodate the undersurface of the foot. Additionally, the device is adaptable to many types and sizes of adult and children's shoes while needing only a limited number of orthotic insert blanks to fit and be useful in a variety of work, sport, dress, and other shoes. The orthopedic insert includes a material that is softened with localized application of heat, and may be re-softened when an initial fit is unsuccessful. The orthopedic insole for the diabetic shoe or the diabetic shoe with the orthopedic insole may be fit and completed during a single office visit to a medical foot specialist.

The present invention provides an orthopedic insole with a therapeutic characteristic for podiatric conditions, particularly those related to diabetes mellitus. The heat-malleable material of the insole can conform and fit to the variations or irregularities of the diabetic foot. Additionally, a pre-molded portion of the insole reduces excessive pronation of the foot, a condition that often leads to foot injury. As a therapeutic device, the insole is capable of relieving foot pain and biomechanically correcting or alleviating misaligned conditions in a foot. The orthopedic insole may help prevent or provide relief from podiatric conditions such as complications from diabetes mellitus, neuromas, hammertoe, heel spurs, bunions, a pronation condition, stress fractures, shin splints, plantar fasciitis, cuboid syndrome, tendonitis, metatarsalgia (ball-of-foot pain), arch pain, or other foot ailments. Diabetics may be more susceptible to foot disease such as ulcers or sores caused by infection and minor injuries that may be avoided or corrected by use of an effective insole. Although the invention can aid in the recovery from a foot ailment, the invention may also serve to prevent the onset or reoccurrence of various foot problems.

The orthopedic insole includes a high-rise heel cup portion of flexible material that absorbs shock during heel strikes, while providing support to the proximal, distal and posterior of the calcaneous or heel bone. The insole also includes a mid-foot portion of flexible material that cooperates with the heel portion to stabilize and support the foot while preventing excessive pronation, and provides a therapeutic characteristic for a podiatric condition.

The deep heel cup portion of the present invention wraps around the heel of the foot and extends above a posterior portion of the heel bone proximal to the Achilles tendon. The mid-foot portion of the insert is continuously coupled to the cupped heel portion. The mid-foot portion includes a curvilinear upper bearing surface and a medial longitudinal arch support to aid in the support of the medial longitudinal arch of the foot. The upper surface of the mid-foot portion includes a minor arch to support the lateral longitudinal arch near the outside of the foot, and a second minor arch to support the transverse arch perpendicular to the medial longitudinal arch and the lateral longitudinal arch of the foot. The mid-foot portion extends from the heel cup towards the heads or anterior ends of the metatarsal bones. The forefoot portion of heat-malleable material is a generally flat or planar section continuously coupled to the mid-foot portion, and contoured around the perimeter to correspond with the sides and distal end of a person's foot. The heat-malleable forefoot material conforms to the shape of the lower surface or undersurface of a user's forefoot when the material is heated to a glass transition temperature at which the forefoot part of the orthopedic insole can be deformed while the user steps down on the insole inside the diabetic shoe. The lower bearing surface of the heat-malleable orthopedic insole may also conform to the inside surface of the shoe when heated and pressed into the shoe. A heat-malleable mid-foot piece may be attached to the mid-foot portion. A heat-malleable rearfoot piece may be attached to at least a portion of the cupped heel portion.

The cupped heel portion and the mid-foot portion coordinate to help realign the rearfoot, avoid overpronation and reduce stress on the Achilles tendon. Excessive pronation renders the gait of a walker or runner less efficient, and is a source of lower extremity pathologies, including muscle tiredness and inflammation, foot and knee joint pain, tendonitis, ligament strain, and even neurological damage. With the full-length insert or insole, the forefoot portion may cushion and reduce stress on the balls and phalangeal area of the foot.

The present invention is an insole that may be inserted into a diabetic shoe or may be an insole that is integrated into the innersole of the diabetic shoe. The actual dimensions of the orthopedic insole, in accordance with the present invention, will vary depending on the size of the foot, the intended use of the shoe, and other factors. The net result of various embodiments of the present invention is a customizable orthopedic insole for a diabetic shoe that protects the sole of the foot, controls pronation, supports the foot, produces a more stable platform on which and in which the foot ambulates, and provides a preventative or curative characteristic for a podiatric condition.

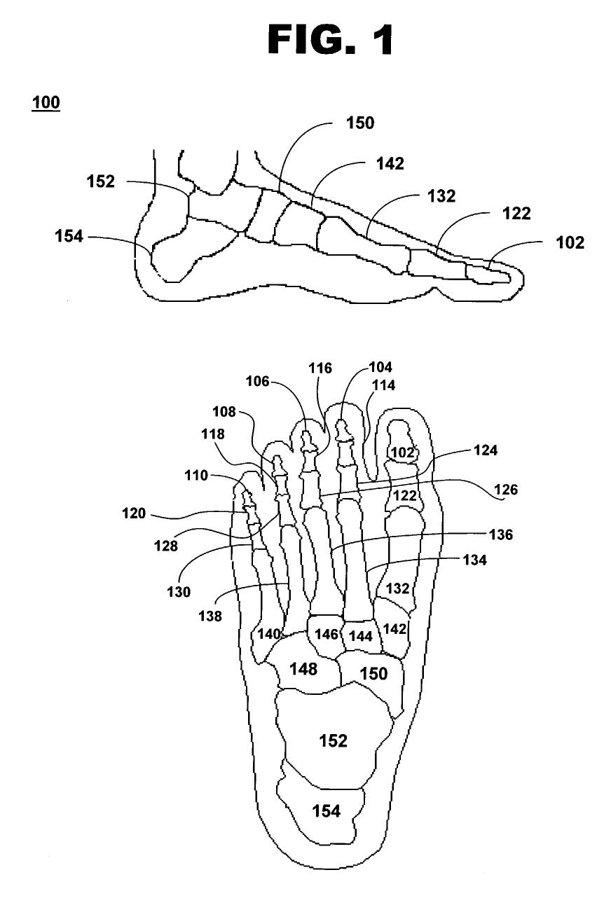

FIG. 1 illustrates a side view and a top view of a human foot at 100. The toes of a human foot are formed by fourteen phalanges. Starting from the inside of the foot, each toe has distal phalanges 102, 104, 106, 108 and 110, middle phalanges 114, 116, 118 and 120, and proximal phalanges 122, 124, 126, 128 and 130. The first phalange or big toe lacks a middle phalange. The forefoot comprises the phalanges and the heads or anterior end of the metatarsals.

The midfoot includes five metatarsals 132, 134, 136, 138 and 140. First metatarsal 132, which is the shortest and thickest of the metatarsal bones, bears the most weight and plays the most important role in propulsion. First metatarsal 132 also provides attachment for several tendons. The more stable second metatarsal 134, third metatarsal 136, and fourth metatarsal 138 are well protected with only minor tendon attachments, and thus are not subjected to strong pulling forces.

The midfoot also includes five of seven tarsal bones: navicular, cuboid, and cuneiform bones. The distal row contains three cuneiforms 142, 144 and 146 and a cuboid 148. The midfoot includes five tarsometatarsal joints, which are among multiple joints within the midfoot itself. Proximally, cuneiforms 142, 144 and 146 articulate with a navicular 150.

A talus 152 and a calcaneus 154 make up the rear or hind portion of the foot. Calcaneus 154 is the largest tarsal bone, and forms the heel. Talus 152 rests on top of it, and forms the pivot for the ankle.

Toe movements take place at joints that are capable of motion in two directions: plantar flexion and dorsiflexion, as well as abduction and adduction. The remainder of the foot has two movements, inversion and eversion, to which joints of the hindfoot and midfoot contribute. These complex movements are combined ordinarily with ankle movements and movements of the fibula and tibia.

Two primary functions of the foot are weight bearing and propulsion, both requiring stability and flexibility. The bones and intervening joints of the foot give flexibility while multiple bones form an arch to support the weight of the body.

The three arches of the foot are the medial longitudinal arch, lateral longitudinal arch, and transverse arch. The inner or medial longitudinal arch, the highest of the arches, comprises calcaneus 154, talus 152, navicular 150, cuneiforms 142, 144 and 146, and first three metatarsals 132, 134 and 136. The outer or lateral longitudinal arch, which is lower and flatter than the medial arch, comprises calcaneus 154, talus 152, cuboid 148, and fifth metatarsal 140. At times, fourth metatarsal 138 is included in the lateral arch. The generally hemispherical arc of the transverse arch comprises cuneiforms 142, 144 and 146, cuboid 148, and the bases of metatarsals 132, 134, 136, 138 and 140. The arches of the foot are maintained by the shapes of the bones and ligaments, and supported by muscles and tendons. The lateral arch, medial arch and transverse arch aid the foot in supporting and distributing the weight of a person. During a heel strike, for example, the force on the heel region may exceed three times the normal weight of the body.

When walking, body weight is first placed on the heel, then forward to the ball of the foot. As body weight is applied to the foot, the arches flatten out slightly to absorb the added pressure, spreading out the force and strain across the bones of the foot evenly. As the foot is lifted before taking another step, the arch springs back into its arched position.

The foot has two primary motions: supination and pronation. Supination is a combination of inward rotation at the ankle, adduction of the hindfoot, inversion of the forefoot, and medial arch elevation. Supination occurs when a heel comes off the ground. Subtalar joint supination involves three simultaneous planes of motion: adduction, inversion, and plantarflexion. As the foot supinates, lateral structures tighten. Continued supination and adduction force may rupture portions of lateral collateral ligaments or avulse these ligaments from their bony attachment sites on the distal fibula, resulting in an ankle sprain.

Subtalar joint pronation involves three simultaneous planes of motion: abduction of a forefoot, eversion of a hindfoot, and dorsiflexion. Because of the close contiguity of the joints involved, pronation is accompanied by eversion of the heel and internal rotation of the leg and hip. In simple terms, pronation is a motion that occurs when the foot lands on the outside edge and the inner arch collapses as far as it can to absorb shock.

Overpronation, the maximum range of motion between pronation and supination, is often cited as a cause of leg and foot problems among runners and a contributor to knee, hip and back pain. While pronation is a normal part of a person's gait, it is understood that excessive pronation may be the source of many lower extremity pathologies, including muscle tiredness and inflammation, foot and knee joint pain, tendonitis, ligament strain, and even neurological damage. Excessive pronation may render the gait less efficient since time and effort is wasted in pronating and supinating.

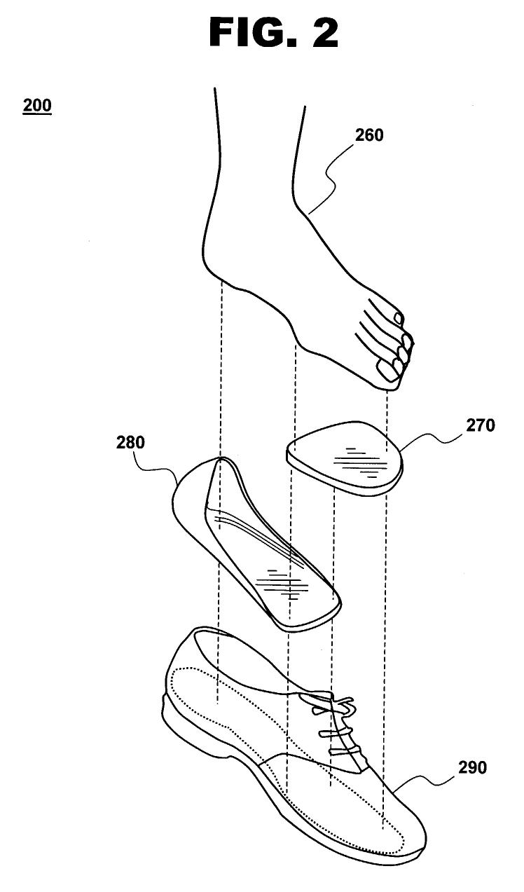

FIG. 2 illustrates a perspective view of a foot and diabetic shoe, the diabetic shoe assembled with a pre-molded insole portion for the heel and mid-foot areas, and a heat-malleable forefront portion, in accordance with the present invention at 200.The diabetic shoe has an orthopedic insole with a cupped heel portion, a mid-foot portion and a heat-malleable forefoot portion. The cupped heel portion has a concave upper bearing surface that extends above a most posterior cephalad portion of a calcaneous. The mid-foot portion is continuously coupled to the cupped heel portion, and has a medial longitudinal arch and a curvilinear upper bearing surface. The heat-malleable forefoot portion is coupled to the mid-foot portion, the forefoot portion having a heat-deformable upper bearing surface.

An exemplary diabetic shoe 290 has a two-part orthopedic insole: a pre-molded piece 280 and a heat-malleable forefoot piece 270 that are coupled to each other to form a smooth and continuous upper-bearing surface of an orthopedic insole for a diabetic shoe. Pre-molded piece 280 has a cupped heel portion and a mid-foot portion whose lower bearing surface substantially conforms to an inside surface of a sole of diabetic shoe 290. Pre-molded piece 280, which is the back portion or approximately the back two-thirds of the entire orthopedic insole, conforms to the contours of the sole of a wearer's foot during use, having a perimeter that encompasses a foot from the heel to a region near the balls of the foot. Pre-moldable piece 280 helps maintain a subtalor joint of a user's foot 260 in an inverted position, and locks the midtarsal joint during ambulation of the foot to reduce pronation and to provide stabilization.

Heat-malleable forefoot piece 270 of an orthopedic insole also includes a forefoot portion having an initially flat or slightly contoured upper bearing surface. The heat-deformable upper bearing surface of the forefoot portion is plastically deformed when the orthopedic insole is heated above a glass transition temperature and compressed by user's foot 260 while the orthopedic insole is above the glass transition temperature. Heat-malleable forefoot potion 270 may comprise a heat-malleable material throughout. Alternatively, the heat malleable forefoot portion may comprise a polymeric lining formed from a heat-malleable material. The heat-malleable forefoot portion may include a layer of heat-malleable material attached to pre-molded piece 280, the layer of heat-malleable material disposed on the upper surface of pre-molded piece 280 and adjacent the undersurface of a user's foot 260. Alternatively, the layer of heat-malleable material may be attached to the lower surface of pre-molded piece 280 and adjacent an inside surface of diabetic shoe 290. Alternatively, the layer of heat-malleable material may be located interior to pre-molded piece 280, as in a laminated layer or as a portion of pre-molded piece 280. Whether the heat-malleable material is located on the upper surface, lower surface, interior to the pre-molded piece or homogeneous throughout, the orthopedic insole may be heated and deformed to substantially conform to the undersurface of a user's foot and to the inside surface of diabetic shoe 290.

The heat-malleable material may include polycaprolactone, polylactide, polyethylene terephthalate, polyglycolide, a thermoplastic polymer, or any combination thereof. The heat-malleable material is selected to have a glass-transition or softening temperature between 45 and 75 degrees centigrade, such that the heat-malleable material can be plastically deformed when the orthopedic insole is heated above a glass transition temperature and compressed by a user's foot while the orthopedic insole is above the glass transition temperature. The heat-malleable material may be heated by inserting the insole into a heating unit such as a container of hot or boiling water, a microwave oven, or a convective oven. Diabetic shoe 290 can be of various designs such as a sports shoe, a children's shoe, a work shoe, a dress shoe, a casual shoe, and a boot. For example, a traditional athletic shoe often has soft-sided uppers that are formed of cloth, vinyl, or other flexible materials that yield outwardly under pressure, thereby providing little inward buttressing around the insole. As another example, the orthopedic insole with pre-molded piece 280 and heat-malleable forefoot piece 270 is integrated into the insole or innersole of a diabetic shoe such as a work boot, a military boot, or a fashion boot. The pre-molded piece 280 and heat-malleable forefoot piece 270 are readily adapted to various sizes and types of shoes. They are designed to protect and be in contact with the bottom of user's foot 260.

The perimeter surface of pre-molded piece 280 and heat-malleable forefoot piece 270 are usually angled to match the inside of the upper where the upper joins the sole of diabetic shoe 290. They also can be integrated into a conventional insole that consists of materials such as synthetic resin foam or elastomer covered with leather, woven fabrics, unwoven fabrics or other materials adhesively bonded thereto. In some embodiments of the present invention, pre-molded piece 280 and heat-malleable forefoot piece 270 are directly attached to the sole of the shoe.

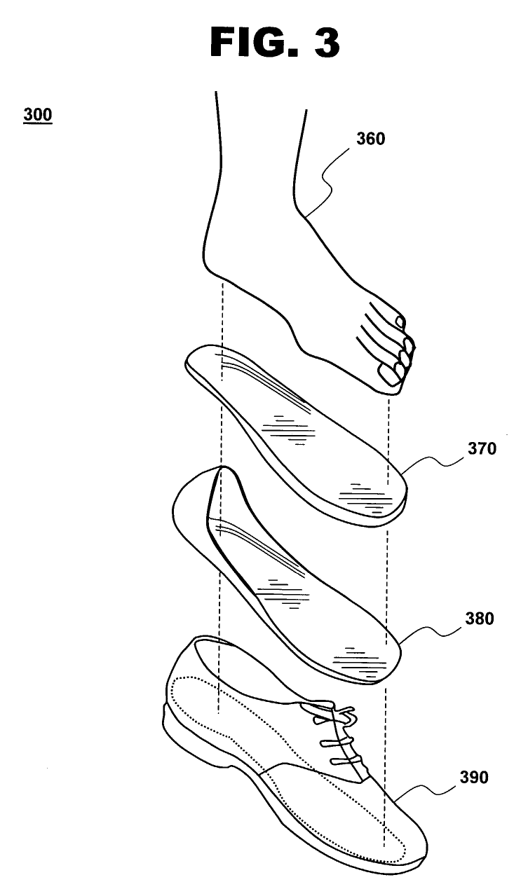

FIG. 3 illustrates a perspective view of a foot and diabetic shoe, the latter assembled with a full-length pre-molded insole portion and a full-length heat-malleable insole portion, in accordance with the present invention at 300. Similar to the diabetic shoe of FIG. 2, this exemplary diabetic shoe 390 has a two-part orthopedic insole, one part being a pre-molded piece 380. The difference is that a heat-malleable piece 370 is a full-length foot size, coupled to the upper surface of pre-molded piece 380, and the heat-malleable material contacts the entire bottom of a wearer's foot 360, rather than just the forefoot. Heat-malleable piece 370 forms a smooth and continuous upper-bearing surface. Alternatively, heat-malleable piece 370 may be attached to the lower surface of pre-molded piece 380. In another embodiment, heat-malleable piece 370 forms is embedded in the interior of pre-molded piece 380. In yet another embodiment, heat-malleable piece 370 extends from the forefoot portion to the mid-foot portion.

FIG. 4 illustrates a perspective view of a full-length pre-molded portion of an orthopedic insole for a diabetic shoe, in accordance with the present invention at 400. An exemplary pre-molded piece 480 of an orthopedic insole 400 for a diabetic shoe includes a cupped heel portion 450 having a concave upper bearing surface 452 and an upwardly concave shape 456 for engaging the heel of a foot, and an upwardly arched mid-foot portion 430 having a medial longitudinal arch support with a curvilinear upper bearing surface 432 for engaging an arch portion of the foot. The upper bearing surface of cupped heel portion 450 may extend above a most posterior cephalad portion of the calcaneous or heel bone.

Pre-molded piece 480 has mid-foot portion 430 formed continuously with cupped heel portion 450 and forefoot portion 420. Forefoot portion 420 has an upper bearing surface 422 and lower bearing surface 424. Mid-foot portion 430 extends from cupped heel portion 450 to an opposite end corresponding to the anterior ends of the metatarsal bones, and from the inner or medial portion to the outer or lateral side of the foot.

Cupped heel portion 450 extends above a posterior portion of a heel bone and is continuously coupled to mid-foot portion 430. Cupped heel portion 450 has a concave upper bearing surface that extends above a most posterior cephalad portion of a calcaneous. Frontal extremities of cupped heel portion 450 may be positioned somewhat more forwardly on the medial side than on the lateral side. Cupped heel portion 450 deforms to conform to the shape of the heel and to provide medial, posterior and lateral support to the calcaneus. A posterior surface of cupped heel portion 450 may engage the heel above the heel bone close to the Achilles tendon. A medial surface and a lateral surface of cupped heel portion 450 may engage the heel bone below the ankle malleolus. The upper edge of cupped heel portion 450 may extend along an arcuate path in a generally descending manner from the Achilles tendon to mid-foot portion 430. Upper bearing surface 452 of cupped heel portion 450 and upper bearing surface 432 of mid-foot portion 430 may be continuously curvilinear, adapted to follow the contours of the plantar surface of the foot. A raised arch area in mid-foot portion 430 provides support for the arches of the foot without collapsing under body weight. Upper bearing surface 452 of cupped heel portion 450 and upper bearing surface 432 of mid-foot portion 430 are contoured to engage the plantar surface of a foot. Lower bearing surface 454 of cupped heel portion 450, lower bearing surface 434 of mid-foot portion 430, and lower bearing surface 424 of forefoot portion 420 may be shaped to substantially conform to an inside surface of a shoe. Similarly, lower bearing surface 454 of cupped heel portion 450 and lower bearing surface 434 of mid-foot portion 430 may be shaped to substantially conform to an inside surface of the sole of a shoe when built into or integrated with the innersole of a shoe.

Pre-molded piece 480 of the orthopedic insole may act simultaneously on the calcaneus and subtalar of the foot. Cupped heel portion 450 may help to stabilize and control the motion of the foot, keeping the heel in its natural state and preventing it from excessively pronating or rolling inward during walking and running, thereby properly aligning the foot and providing better shock absorption and stress distribution.

Cupped heel portion 450 and mid-foot portion 430 may cooperate to provide a therapeutic characteristic for a podiatric condition, which may include plantar fasciitis or another medical condition such as cuboid syndrome, a neuroma, hammertoe, a bunion, a pronation condition, tendonitis, or a foot ailment. Other podiatric conditions may include fat pad atrophy, heel spurs, metatarsalgia, diabetic foot, hyperkeratosis, Morton's neuroma, plantar pain from arthritis or peak shock load, sore heels, sore knees, shin splints, Sever's disease, calcaneal apophysitis, bursitis, achilles tendonitis, and elongated metatarsals. The cupped heel portion 450 and mid-foot portion 430 are typically made of a flexible and moldable material such a neoprene rubber, a silicone rubber, an elastomer, a polymeric material, a urethane, polyethylene terephthalate, a viscoelastic material, a silicone gel, or any combination thereof.

In one embodiment of the orthopedic insole being described, pre-molded forefoot portion 420 extends from the forward end of mid-foot portion 430 to the end of forefoot portion 420 corresponding to the metatarsal heads of a user's foot, and from a medial side to a lateral side of the foot. Forefoot portion 420 includes a heat-malleable material and has a heat-deformable upper bearing surface. Forefoot portion 420 is continuously coupled to mid-foot portion 430 and extends from the front of mid-foot portion 430 to a region corresponding with the distal end of the foot while comfortably encompassing the bottoms of the toes. Forefoot portion 420 may reduce stress on the balls of the foot, and aid in distributing ambulatory stresses into the front portion of the foot.

Pre-molded piece 480 of the orthopedic insole may be relatively thick in cupped heel portion 450 under and around the heel of the foot, and relatively thin and flexible near its upper and lateral edges. Pre-molded piece 480 may be relatively thick at the arched regions of mid-foot portion 430, particularly in the region under the medial longitudinal arch of the foot, and relatively thin near the sides. Alternatively, pre-molded piece 480 may be relatively thin and initially flat or slightly contoured in forefoot portion 420. The thickness or thinness is dependent on the dimensions of a, heat-malleable forefront piece that is coupled to mid-foot portion 430. The size of the entire insole is selected to accommodate a particular shoe size. The dimensions of the insole and in particular, pre-molded piece 480, are determined to provide a proper fit. Pre-molded piece 480 has a seamless surface with contours to provide structural stability and foot support.

The lightweight material of pre-molded piece 480 provides dynamic control as well as static balance. In one embodiment, the lower layer of orthopedic insole 400 is made from a flexible material that can cushion and absorb the shock from heel strike on orthopedic insole 400. Pre-molded piece 480 can be formed from a substantially flexible, resiliently compressible cushioning material having an upper surface for engaging a plantar surface of a foot and a bottom surface for engaging a sole of a shoe. Pre-molded piece 480 can also use a semi-rigid, injection moldable material. The cupped heel portion and the mid-foot portion may be formed from a flexible material. The durometer value of the flexible material may extend from a value less than 20 to a value in excess of 70. The flexible material includes a neoprene rubber, a silicone rubber, an elastomer, a polymeric material, a urethane, polyethylene terephthalate, a viscoelastic polymer, a silicone gel, and combinations thereof.

The flexible and shock-absorbing polymeric material is a lightweight and durable thermoplastic such as polyethylene or cross-linked ethylene vinyl acetate foam, cross-linked polyethylene, poly(ethylene-vinyl acetate), polyvinyl chloride, an acrylic, synthetic and natural latex rubbers, block polymer elastomers, thermoplastic elastomers, polystyrene, ethylenepropolene rubbers, silicone elastomers, polystyrene, polyurea or polyurethane, a polyurethane foam, an elastomeric foam, a non-foam elastomer, or any combination thereof.

The flexible material may comprise a gripping characteristic to allow the shoe insert to firmly engage a heel and midfoot, and to provide proximal, posterior and lateral support when engaged with the calcaneous. Pre-molded piece 480 may have a texture embossed on the upper bearing surface of at least the cupped heel portion to improve the gripping characteristic. The gripping characteristic may be due, at least in part, by mechanical coupling of the heel bone to the orthopedic insole, and enhanced by insertion of the foot into a shoe with the orthopedic insert. Additional gripping capability is generated by friction between the calcaneous and the orthopedic insole, and is affected by the materials, shape, and texture of the insole.

The material used in pre-molded piece 480 is a compression-resistant, deformable material that provides shock attenuation and support for the foot.

Additional reinforcing support members may be built into the pre-molded piece 480. For example, a rim region of harder material may surround the base of the cupped heel portion. Reinforcing support members may be built into the cupped heel portion of the insert to provide additional support of the calcaneous, using, for example, semi-circular rods of high strength, resilient material extending around the back and sides of the heel, or upwards from the base of the cupped heel portion towards the ankle. Regions of soft, gel-like material may be incorporated into select regions of the insert, such as directly underneath the fat pad of the foot where heels may bruise and bone spurs may occur.

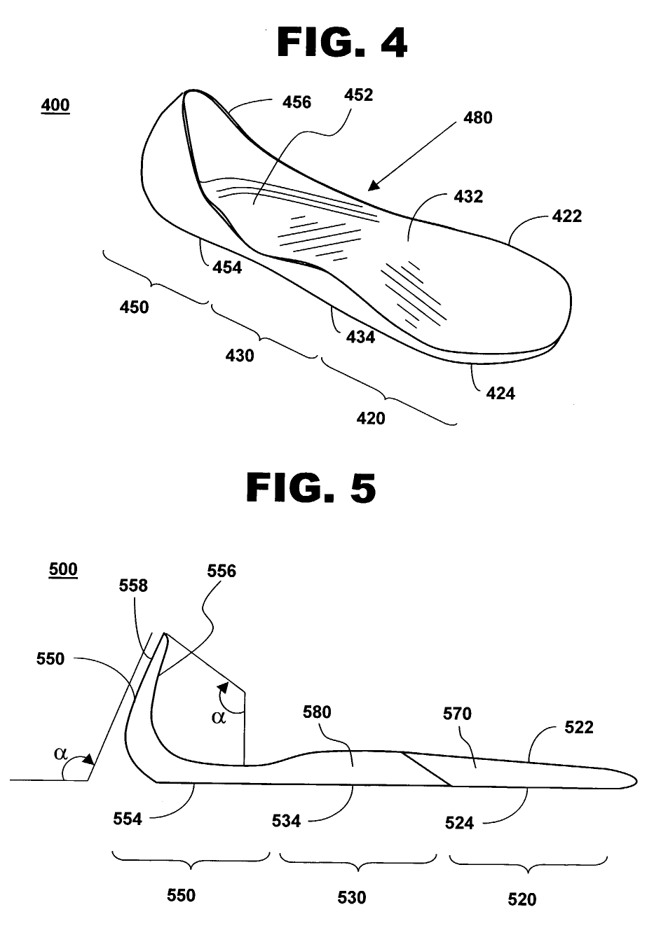

FIG. 5 illustrates a cross-sectional view of an orthopedic insole with a forefoot heat-malleable piece and a pre-molded piece of two-thirds length with mid-foot and cupped heel portions, in accordance with the present invention at 500. In this embodiment of the present invention, pre-molded piece 580 comprises a mid-foot portion 530 and a cupped heel portion 550, with lower bearing surfaces 534 and 554, respectively. Heat-malleable piece 570 comprises a forefoot portion 520 of the orthopedic insole with an upper bearing surface 522 and a lower bearing surface 524.

Lower bearing surfaces 524, 534 and 554 may be contoured to conform to an inside surface of a shoe and may have some texture, embossed patterns or other indenting or protruding features, although the surfaces generally are flat and continuous with respect to one another.

The rear part of cupped heel portion 550 opens toward mid-foot portion 530, the heel cup being designed and dimensioned for adapting to the calcaneus. Cupped heel portion 550 may be continuously curved. An inner arcuate portion 556 and an outer arcuate portion 558 of cupped heel portion 550 above the calcaneous may be angled forwardly and upwardly and accorded a heel cup angle alpha (a), the heel cup angle alpha being measured by an arc sweeping from the base of the upwardly concave cupped heel portion 550 to the top of inner arcuate portion 556. Alternatively, heel cup angle alpha may be measured by an angle corresponding to a line essentially parallel to lower bearing surface 554 of cupped heel portion 550 and a line essentially tangential to the top of outer arcuate portion 558, with a larger heel cup angle corresponding to a fuller heel cup. The heel cup angle of the currently preferred embodiment may be greater than 60 degrees, and preferably greater than 90 degrees.

A larger heel cup angle provides more support and stability for the calcaneous, cooperating with mid-foot portion 530 to invert the subtalor joint of a foot to a position of slight inversion while walking or running.

The medial, posterior, and lateral portions of the heel cup may hold the vertical axis of the calcaneus essentially coaxial with the axis of the leg. The longitudinal axis of the heel cup and mid-foot portions are oriented toward the fifth metatarsus of the foot so as to orient likewise the calcaneus. The mid-foot portion has a curvilinear upper bearing surface to support the subtalar. The upper surface of the orthopedic insole is contoured to engage and cradle the plantar surface of a person's foot, and the bottom surface may be generally flat and planar, or shaped to conform to the inner surface of a shoe.

Cupped heel portion 550 permits limited freedom of movement of the heel relative to the mid-foot portion when the insole is worn. The bottom region of the heel cup may be thicker to absorb the primary force of a heel strike. Reinforcement support members may optionally be embedded and secured into the heel cup to provide additional support for the calcaneous. Regions of softer, pliable material or detents may be formed in the bottom region of the heel cup to provide comfort and relief from heel spurs, for example, or atrophy of the fat pad.

Forefoot heat-malleable piece 570 with forefoot upper bearing surface 522 is coupled to pre-molded piece 580. The heat-malleable forefoot portion may comprise a heat-malleable material such as polycaprolactone, polylactide, polyethylene terephthalate, polyglycolide, a thermoplastic polymer, or any combination thereof. The heat-malleable piece is attached to pre-molded piece 580 by glue, an adhesive, or some other coupling mechanism. In this and other embodiments of the invention that are described herein, a pharmaceutical compound such as a foot odor control compound, an anti-inflammatant for reducing inflammation, vascular endothelial growth factor (VEGF) for stimulating new blood vessel growth, a wound-healing agent, a cortical steroid, or a therapeutic agent may be included in the heat-malleable material of at least the forefoot portion. The heat-deformable upper bearing surface 522 of forefoot portion 520 is plastically deformed when the orthopedic insole is heated above a glass transition temperature of the heat-malleable material and is compressed by a user's foot while the orthopedic insole is above the glass transition temperature. That temperature is typically between 45 and 75 degrees centigrade.

In another embodiment of the present invention, the heat-malleable piece extends over the mid-foot portion 530 and is continuously coupled to heat-malleable piece 570. The heat-malleable mid-foot piece may be attached to the upper bearing surface or the lower bearing surface of the mid-foot portion. In another embodiment of the present invention, the pre-molded piece and the heat-malleable piece are both full-length, with the heat-malleable piece on the upper surface of a full-length molded piece. Another embodiment has a full-length pre-molded piece, and at least part of the heat-malleable material is coupled to the lower bearing surface of the pre-molded piece. In another embodiment, a heat-malleable rearfoot piece is attached to at least a portion of the cupped heel portion, such as the upper bearing surface of the cupped heel portion.

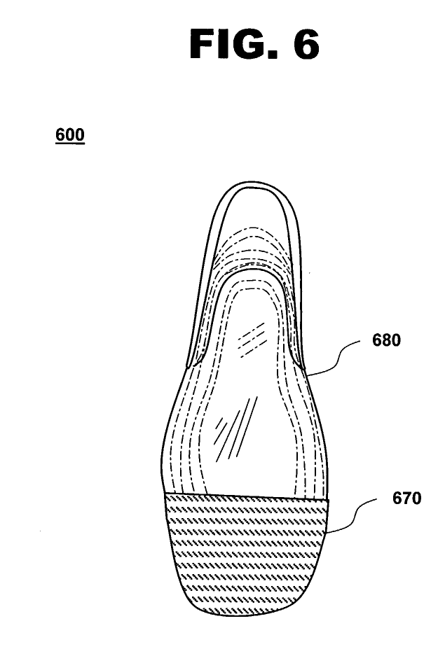

FIG. 6 illustrates a perspective view of an inside of an orthopedic insole for a diabetic shoe, in accordance with the current invention at 600. Orthopedic insole 600 with a heat-malleable portion 670 and a pre-molded portion 680 may have upper or bottom surfaces that are either smooth or are embellished with various patterns and textures. The flexible material of the pre-molded portion 680 that is textured can have a gripping characteristic to provide proximal, posterior and lateral support when engaged with the calcaneous of a foot. The texture may be particularly effective on the upper bearing surface of the heel cup of a pre-molded piece 680, which helps to effectively engage the heel and redistribute stresses. Texture surfaces may enhance contact with the foot or the sole of a shoe. Textured surfaces such as deep waffle or honeycomb patterns on the lower bearing surface of orthopedic insole 600 may enhance its shock-absorbing qualities.

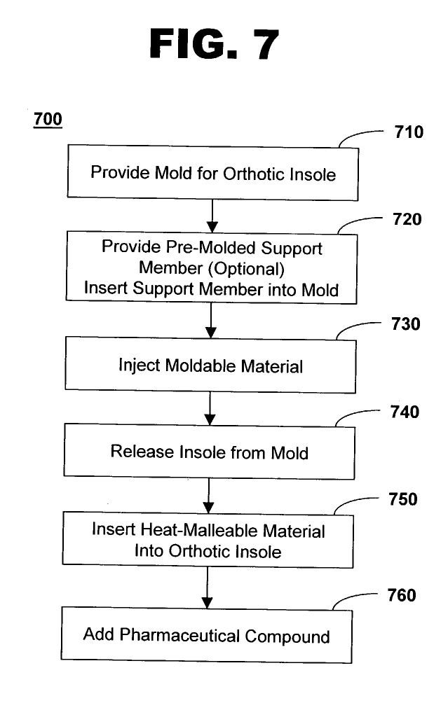

FIG. 7 is a flow diagram of a method of manufacturing an orthopedic insole for a diabetic shoe, in accordance with the present invention at 700. Insole manufacturing method 700 begins by providing an orthopedic insert mold as seen at block 710. The mold has a cavity for a cupped heel portion, a mid-foot portion, and a forefoot portion. A forefoot portion is coupled to the mid-foot portion.

The orthopedic insole that results from the manufacturing process has a cupped heel portion with a concave upper bearing surface that extends above a most posterior cephalad portion of a calcaneous of a foot. The concave upper bearing surface of the cupped heel portion has a heel cup angle of at least 60 degrees. The mid-foot portion is continuously coupled to the heel portion, the mid-foot portion having a medial longitudinal arch and a curvilinear upper bearing surface. A forefoot portion has a heat-deformable upper bearing surface, the upper bearing surface having the general outline around the toe or distal end of a foot.

In an exemplary embodiment of the present invention, an optional support member can be added to the moldable material to provide additional structure and reinforcement, as seen as block 720. For example, a rim region of harder material can be used to surround the base of the cupped heel portion. Another example is where reinforcing support members are built into the cupped heel portion of the insert to provide additional support of the calcaneous, using semi-circular rods of high strength, resilient material extending around the back and sides of the heel, or upwards from the base of the cupped heel portion towards the ankle. The optional pre-molded support member can be inserted into the orthopedic insole mold prior to the injection of a molding compound.

A moldable material is injected into the orthopedic insole mold, as seen at block 730. The injection-molding compound may include a neoprene rubber, a silicone rubber, an elastomer, a polymeric material, a urethane, polyethylene terephthalate, a viscoelastic material, a silicone gel, and any combination thereof. The compound may be cured or treated to form the flexible material, as is known in the art. When a pre-molded support member is provided, the member is inserted into the insert mold prior to injecting the injection-molding compound.

The orthopedic insole is released from the orthopedic insole mold, as seen at block 740. At this point or later, an optional absorbing material may be attached to at least a portion of the upper bearing surface of the pre-molded part of the orthopedic insole.

A heat-malleable material then is inserted into the orthopedic insole, as seen at block 750. The heat-malleable material is inserted into at least the forefoot portion of the orthopedic insole to functionalize the heat-deformable upper bearing surface. The heat-malleable forefoot portion is coupled to the mid-foot portion. An adhesive, glue or other attaching method can be used to attach the heat-malleable forefoot portion to the mid-foot portion. As an alternative, the heat-malleable material can be inserted into the areas of the mid-foot and rearfoot portions, as well as the forefoot portion. In this configuration, the heat-malleable material is coupled to the upper bearing surface of the injection-molded part of the insole. In other embodiments, the heat-malleable material is placed below the forefoot portion, the mid-foot portion, the rearfoot portion, or combinations thereof.

The upper bearing surface of the cupped heel portion, the curvilinear upper bearing surface of the mid-foot portion, and the heat-deformable upper bearing surface substantially conform to an undersurface of a foot. The heat-deformable upper bearing surface of the forefoot portion is manufactured from a material that may be plastically deformed when the orthopedic insole is heated above a glass transition, which is typically between 45 and 75 degrees centigrade.

A pharmaceutical compound is added optionally to the orthopedic insole, as seen at block 760. The pharmacology may be added during the preparation of the heat-malleable material or imbibed by local pharmacies. The heat-malleable material can be heated to aid in the imbibing of the pharmaceutical therapy or otherwise enhanced to aid in the uptake of the pharmaceutical compounds. The heat-malleable material is then reduced to ambient temperature to lock in the compounds. A polymeric lining comprised of heat-malleable material may be designed to release the pharmacology when in contact with the foot through absorption processes, enhanced by the slightly elevated temperature of the foot.

The malleable lining can be enhanced by the addition of additives that control foot odor, and are released with increase in temperature from ambient. An absorbing material optionally may be attached to at least a portion of the upper bearing surface of the orthopedic insole.

When at an podiatrist's office or other suitable office or home setting, the orthopedic insole may be heated by inserting the insole into a heating unit such as boiling water, hot water, a microwave oven, a convective oven to soften the heat-malleable portion and allow the user to custom fit the orthopedic insole to the bottom surface of the foot. The lower bearing surface of the orthopedic insole may be substantially conformed to an inside surface of a shoe or article of footwear.

Alternately, other forms of manufacturing and production may be used as is known in the art, such as thermoforming, cutting or stamping.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed as invention is:

1. An orthopedic insole for a diabetic shoe, comprising:
a cupped heel portion, the cupped heel portion having a concave upper bearing surface that extends above a most posterior cephalad portion of a calcaneous;
a mid-foot portion continuously coupled to the cupped heel portion, the mid-foot portion having a medial longitudinal arch and a curvilinear upper bearing surface; and
a heat-malleable forefoot portion coupled to the mid-foot portion, the forefoot portion having a heat-deformable upper bearing surface.

2. The orthopedic insole of claim 1 wherein the cupped heel portion and the mid-foot portion cooperate to invert a subtalor joint of a foot to a position of inversion and to lock a midtarsal joint of the foot during ambulation to reduce pronation and provide stabilization.

3. The orthopedic insole of claim 1 wherein the concave upper bearing surface of the cupped heel portion comprises a heel cup angle of at least 90 degrees.

4. The orthopedic insole of claim 1 wherein the mid-foot portion extends from the cupped heel portion to an opposite end corresponding to an anterior end of a metatarsal bone.

5. The orthopedic insole of claim 1 wherein the upper bearing surface of the cupped heel portion, the curvilinear upper bearing surface of the mid-foot portion, and the heat-deformable upper bearing surface of the forefoot portion substantially conform to an undersurface of a foot.

6. The orthopedic insole of claim 1 wherein the cupped heel portion and the mid-foot portion are formed from a flexible material.

7. The orthopedic insole of claim 6 wherein the flexible material is selected from the group consisting of a neoprene rubber, a silicone rubber, an elastomer, a polymeric material, a urethane, polyethylene terephthalate, a viscoelastic material, a silicone gel, and combinations thereof.

8. The orthopedic insole of claim 6 wherein the flexible material comprises a gripping characteristic to provide proximal, posterior and lateral support when engaged with the calcaneous.

9. The orthopedic insole of claim 1 wherein the heat-malleable forefoot portion comprises a polymeric lining formed from a heat-malleable material.

10. The orthopedic insole of claim 1 wherein the heat-malleable forefoot portion comprises a layer of heat-malleable material disposed on an upper surface of the orthopedic insole, the upper surface adjacent an undersurface of a foot.

11. The orthopedic insole of claim 1 wherein the heat-malleable forefoot portion comprises a layer of heat-malleable material disposed on a lower surface of the orthopedic insole, the lower surface adjacent an inside surface of the diabetic shoe.

12. The orthopedic insole of claim 1 wherein the heat-malleable forefoot portion comprises a layer of heat-malleable material interior to the forefoot portion.

13. The orthopedic insole of claim 12 wherein the heat-malleable material comprises a material selected from the group consisting of polycaprolactone, polylactide, polyethylene terephthalate, polyglycolide, and a thermoplastic polymer.

14. The orthopedic insole of claim 12 wherein the heat-malleable material comprises a pharmaceutical compound in at least the forefoot portion.

15. The orthopedic insole of claim 14 wherein the pharmaceutical compound comprises a foot odor control compound.

16. The orthopedic insole of claim 14 wherein the pharmaceutical compound is selected from the group consisting of an anti-inflammatant, vascular endothelial growth factor, a wound-healing agent, a cortical steroid, and a therapeutic agent.

17. The orthopedic insole of claim 1 wherein the heat-deformable upper bearing surface of the forefoot portion is plastically deformed when the orthopedic insole is heated above a glass transition temperature and compressed by a user's foot while the orthopedic insole is above the glass transition temperature.

18. The orthopedic insole of claim 17 wherein the glass transition temperature is between 45 and 75 degrees centigrade.

19. The orthopedic insole of claim 1 wherein the cupped heel portion, the mid-foot portion, and the forefoot portion cooperate to provide a therapeutic characteristic for a podiatric condition.

20. The orthopedic insole of claim 19 wherein the podiatric condition is selected from the group consisting of a diabetes mellitus complication, neuroma, a hammertoe, a heel spur, a bunion, a pronation condition, a stress fracture, shin splints, plantar fasciitis, cuboid syndrome, tendonitis, metatarsalgia, arch pain, and a foot ailment.

21. The orthopedic insole of claim 1 further comprising:
a lower bearing surface substantially conforming to an inside surface of a shoe.

22. The orthopedic insole of claim 1 further comprising:
a texture embossed on the upper bearing surface of at least the cupped heel portion.

23. The orthopedic insole of claim 1 further comprising:
a reinforcing support member built into the orthopedic insole.

24. The orthopedic insole of claim 1 further comprising:
a heat-malleable mid-foot piece attached to the mid-foot portion.

25. The othopedic insole of claim 1 further comprising:
a heat-malleable rearfoot piece attached to at least a portion of the cupped heel portion.

26. A diabetic shoe with an orthopedic insole, the orthopedic insole comprising:
a cupped heel portion, the cupped heel portion having a concave upper bearing surface that extends above a most posterior cephalad portion of a calcaneous;
a mid-foot portion continuously coupled to the cupped heel portion, the mid-foot portion having a medial longitudinal arch and a curvilinear upper bearing surface; and
a heat-malleable forefoot portion coupled to the mid-foot portion, the forefoot portion having a heat-deformable upper bearing surface.

27. The diabetic shoe of claim 24 wherein the orthopedic insole is integrated into the innersole of the diabetic shoe.

28. A method of manufacturing an orthopedic insole for a diabetic shoe comprising:
providing an orthopedic insole mold with a cavity for a cupped heel portion, the cupped heel portion having a concave upper bearing surface that extends above a most posterior cephalad portion of a calcaneous; a mid-foot portion continuously coupled to the heel portion, the mid-foot portion having a medial longitudinal arch and a curvilinear upper bearing surface; and a forefoot portion coupled to the mid-foot portion, the forefoot portion having a heat-deformable upper bearing surface;
injecting an injection-molding compound into the orthopedic insole mold; and
releasing the orthopedic insole from the orthopedic insole mold.

29. The method of claim 28 wherein the injection-molding compound is selected from the group consisting of a neoprene rubber, a silicone rubber, an elastomer, a polymeric material, a urethane, polyethylene terephthalate, a viscoelastic material, a silicone gel, and combinations thereof.

30. The method of claim 28 further comprising:
providing a pre-molded support member; and
inserting the pre-molded support member into the orthopedic insole mold prior to injecting the injection-molding compound.

31. The method of claim 28 further comprising:
inserting a heat-malleable material into at least the forefoot portion of the orthopedic insole.

32. The method of claim 28 further comprising:
adding a pharmaceutical compound to the orthopedic insole.
